(19) United States
(12) Patent Application Publication
(10) Pub. No.: US 2025/0174357 A1
(43) Pub. Date: May 29, 2025

(54) Machine-Learning Models for Prognosing Outcomes For Hypertrophic Cardiomyopathy (HCM)

(71) Applicant: Bristol-Myers Squibb Company, Princeton, NJ (US)

(72) Inventors: Arnaud Bastien, Princeton, NJ (US); Amy Sehnert, Princeton, NJ (US); Joan Buenconsejo, Princeton, NJ (US); Gengyuan Liu, Lawrenceville, NJ (US); Sazedul Alam, Lawrenceville, NJ (US); Kevin Elgui, Paris (FR); Paul Trichela, Paris (FR); Mathieu Andreux, Paris (FR); Auriane Riou, Paris (FR); Christian Esposito, Paris (FR); Maria Teresa Telenczuk, Paris (FR); Antoine Olivier, Paris (FR); Antoine Simon, Paris (FR); Benoît Sauty De Chalon, Paris (FR); Valérie Ducret, Paris (FR); Maxime Touzot, Paris (FR); Félix Balazard, Paris (FR)

(73) Assignee: Bristol-Myers Squibb Company, Princeton, NJ (US)

(21) Appl. No.: 18/956,668

(22) Filed: Nov. 22, 2024

(30) Foreign Application Priority Data
Nov. 24, 2023 (EP) 23307056.4
Nov. 30, 2023 (EP) 23307108.3

(51) Int. Cl. G16H 50/20; G16H 10/60

(52) U.S. Cl. CPC G16H 50/20; G16H 10/60

(57) ABSTRACT

A computer-implemented method includes receiving, from one or more sources, multidimensional medical data for a patient, extracting features from the multidimensional medical data, and processing, using one or more ML medical prognosis models, the features to predict a probability of experiencing a progression of HCM by a threshold date. The one or more models trained using a training process including, for each of a plurality of patients, obtaining corresponding baseline training data comprising baseline multidimensional medical data spanning multiple different modalities, and obtaining corresponding follow-up training data including follow-up multidimensional medical data spanning multiple different modalities and collected after the baseline training data and a corresponding ground-truth clinical outcome. The training process including training the one or more models on the baseline training data and the follow-up training data to teach the one or more models to learn how to predict the corresponding ground-truth clinical outcomes.

### CROSS REFERENCE TO RELATED APPLICATIONS

This U.S. patent application claims priority under 35 U.S.C. § 119 from European Patent Application No. EP-23307108.3, filed on Nov. 30, 2023, and from European Patent Application No. EP-23307056.4, filed on Nov. 24, 2023. The disclosures of these prior applications are considered part of the disclosure of this application and are hereby incorporated by reference in their entireties.

### TECHNICAL FIELD

This disclosure relates to machine-learning (ML) models for prognosing outcomes for hypertrophic cardiomyopathy (HCM).

### BACKGROUND

Hypertrophic cardiomyopathy (HCM) is a chronic heart condition that causes the heart muscle to thicken and stiffen. HCM is one of the most common inherited cardiac disorders with an estimated prevalence of 1 in 200 to 1 in 500 persons. It is characterized by left ventricular hypertrophy (LVH) in the absence of another cardiac, systemic, or metabolic trigger such as hypertension. In genetic HCM, the disease is inherited in an autosomal dominant pattern and caused by a mutation in sarcomere protein genes. In idiopathic HCM, the disease may not have known genetic cause. In general, HCM causes alterations of the structure of the heart affecting its function.

Cardiac myosin inhibitors have recently emerged as a new treatment option for patients with HCM and are considered to have a disease-modifying potential. Trials for the effectiveness of cardiac myosin inhibitors have focused on symptomatic New York Heart Association (NYHA) class II and class III HCM patients. As the cardiac myosin inhibitors treatment is disease specific, it can be expected to prevent asymptomatic patients from progression to symptomatic disease.

### SUMMARY

One aspect of the disclosure provides a computer-implemented method executed on data processing hardware that causes the data processing hardware to perform operations. The operations including receiving, from one or more sources, multidimensional medical data for a patient, extracting features from the multidimensional medical data, and processing, using one or more machine-learning (ML) medical prognosis models, the extracted features to predict a probability of experiencing a progression of hypertrophic cardiomyopathy (HCM) in the patient by a threshold date. The one or more ML medical prognosis models trained using a training process including obtaining, for each of a plurality of patients, corresponding baseline training data including baseline multidimensional medical data spanning multiple different modalities, and obtaining, for each of the plurality of patients, corresponding follow-up training data including follow-up multidimensional medical data spanning multiple different modalities and collected after the baseline training data, and a corresponding ground-truth clinical outcome. The training process also including training the one or more ML medical prognosis models on the baseline training data and the follow-up training data to teach the one or more ML medical prognosis models to learn how to predict the corresponding ground-truth clinical outcomes.

Implementations of the disclosure may include one or more of the following optional features. In some examples, the one or more ML medical prognosis models include one or more models selected from a group consisting of: a survival model, a neural network, a convolutional neural network (CNN), an attention-based neural network, a generative neural network, an autoencoder, a variational autoencoder (VAE), a regression model, a linear model, a non-linear model, a support vector machine, a decision tree model, a random forest model, an ensemble model, a Bayesian model, a naïve Bayes model, a k-means model, a k-nearest neighbors model, a principal component analysis, a Markov model, and any combinations thereof. In some implementations, the regression model includes a multivariate survival model or a multivariate temporal response function model (mTRF model), wherein the mTRF model includes independent variables corresponding to the multidimensional medical data. In some implementations, the generative neural network includes a conditional generative adversarial network (cGAN), the cGAN includes one or more conditions corresponding to the multidimensional medical data.

In some implementations, the baseline multidimensional medical data is collected for the patient within a threshold time period from an index date corresponding to when the patient was diagnosed with HCM. In some examples, the multidimensional medical data includes at least one of medical imaging data, a cardiac measurement, clinical data, electrocardiogram data, a laboratory test result, genomic data, or a functional test result. In some implementations, the predicted probability includes a predicted probability in the patient experiencing a cardiovascular event including at least one of a cardiovascular-related hospitalization, a new diagnosis of atrial fibrillation, an episode of heart failure requiring treatment, an episode of lethal ventricular arrhythmia leading to sudden cardiac arrest, an appropriate implantable cardioverter defibrillator shock, a transient ischemic attack, a stroke, death, an acute myocardial infarction, a worsening in pVO2, or a worsening in LVOT gradient.

In some examples, the patient has a New York Health Association (NYHA) class assessment of class I, and the predicted probability has a predicted probability of the HCM in the patient progressing to a NYHA class assessment of class II or higher by the threshold date. In other examples, the patient has a New York Health Association (NYHA) class assessment of early-stage class II, and the predicted probability includes a predicted probability of the HCM in the patient progressing to a NYHA class assessment of class III or higher by the threshold date. In more examples, the predicted probability includes a predicted probability of at least one of the HCM in the patient transitioning from non-obstructed HCM to obstructed HCM by the threshold date, or initiation of an HCM therapy by the threshold date.

In some implementations, the operations also include receiving follow-up multidimensional medical data for the patient, the follow-up multidimensional medical data collected for the patient during a follow-up visit, extracting follow-up features from the follow-up multidimensional medical data, and processing, using one or more trained ML medical prognosis models, the extracted features to predict the probability of experiencing the progression of HCM by the threshold date is further based on processing, using the one or more trained ML medical prognosis models, the extracted follow-up features. In other examples, the operations also include determining that the predicted probability satisfies a threshold probability value, and, based on determining that the predicted probability satisfies the threshold probability value, selecting the patient for inclusion in a clinical trial. In still other examples, the operations also include determining that the predicted probability satisfies a threshold probability value, and, based on determining that the predicted probability satisfies the threshold probability value, treating the patient with a cardiac myosin inhibitor.

Another aspect of the disclosure provides a computer-implemented method executed on data processing hardware that causes the data processing hardware to perform operations. The operations include, for each particular patient of a plurality of patients diagnosed with hypertrophic cardiomyopathy (HCM) and satisfying an inclusion criterion: obtaining corresponding baseline training data includes baseline multidimensional medical data spanning multiple different modalities and collected for the particular patient within a threshold time period from a corresponding index date assigned to the particular patient; and obtaining corresponding follow-up training data including follow-up multidimensional medical data spanning multiple different modalities and collected for the particular patient after the corresponding index date, and a corresponding ground-truth clinical outcome. The operations also include training one or more machine-learning (ML) medical prognosis models on the corresponding baseline training data and the corresponding follow-up training data to teach the one or more ML medical prognosis models to predict the corresponding ground-truth clinical outcomes.

In some examples, the multidimensional medical data includes at least one of medical imaging data, a cardiac measurement, clinical data, electrocardiogram data, a laboratory test result, genomic data, or a functional test result. In some implementations, the predicted probability includes a predicted probability in the patient experiencing a cardiovascular event including at least one of a cardiovascular-related hospitalization, a new diagnosis of atrial fibrillation, an episode of heart failure requiring treatment, an episode of lethal ventricular arrhythmia leading to sudden cardiac arrest, an appropriate implantable cardioverter defibrillator shock, a transient ischemic attack, a stroke, death, an acute myocardial infarction, a worsening in pVO2, or a worsening in LVOT gradient.

In some examples, the patient has a New York Health Association (NYHA) class assessment of class I, and the predicted probability has a predicted probability of the HCM in the patient progressing to a NYHA class assessment of class II or higher by the threshold date. In other examples, the patient has a New York Health Association (NYHA) class assessment of early-stage class II, and the predicted probability includes a predicted probability of the HCM in the patient progressing to a NYHA class assessment of class III or higher by the threshold date. In more examples, the predicted probability includes a predicted probability of at least one of the HCM in the patient transitioning from non-obstructed HCM to obstructed HCM by the threshold date, or initiation of an HCM therapy by the threshold date.

In some implementations, training the one or more ML medical prognosis models on the corresponding baseline training data and the corresponding follow-up training data obtained for a particular patient includes, for each respective modality of the multiple different modalities: extracting, from the corresponding baseline training data, baseline features associated with the respective modality; extracting, from the follow-up corresponding training data, follow-up features associated with the respective modality; and training, a respective modality-specific ML medical prognosis model, on the baseline features and the follow-up features associated with the respective modality.

In some examples, for at least one particular patient of the plurality of patients: obtaining the corresponding baseline training data and the corresponding follow-up training data includes accessing, via a federated data access technique, a local storage device that stores the corresponding baseline training data and the corresponding follow-up training data for the particular patient, the local storage device controlled by an owner of the corresponding baseline training data and the corresponding follow-up training data; and training the one or more ML medical prognosis models on the corresponding baseline training data and the corresponding follow-up training data includes training the one or more prognosis models by processing the corresponding baseline training data and the corresponding follow-up training accessed from the local storage device locally on a respective worker node controlled with the owner of the corresponding baseline training data and the corresponding follow-up training data.

### DESCRIPTION OF DRAWINGS

FIG. 1 is a schematic view of an example prognosing system for predicting a progression of hypertrophic cardiomyopathy (HCM) in a patient.

FIGS. 2A and 2B are a schematic view of an example artificial neural network (ANN) for predicting progression of HCM in a patient.

FIG. 3 is a schematic view of an example convolutional neural network (CNN) for predicting progression of HCM in a patient.

FIG. 4 is a schematic view of an example generative adversarial network (GAN) for predicting progression of HCM in a patient.

FIG. 5 is a schematic view of an example variational auto encoder (VAE) for predicting progression of HCM in a patient.

FIG. 6 is a schematic view of an example fusion architecture.

FIG. 7 is a schematic view of another example fusion architecture.

FIG. 8 is a schematic view of an example training process for training an ML medical prognosis model for predicting progression of HCM in a patient.

FIG. 9 is a schematic view of another example training process for training an ML medical prognosis model for predicting progression of HCM in a patient.

FIG. 10 is a schematic view of an example training process for training a plurality of ML medical prognosis models for predicting progression of HCM in a patient.

FIG. 11 is a schematic view of another example training process for training a plurality of ML medical prognosis models for predicting progression of HCM in a patient.

FIG. 12 is a schematic view of an example data pre-processing process for generating a training data set for training an ML medical prognosis model for predicting progression of HCM in a patient.

FIG. 13 is a schematic view of an example process for generating an ML medical prognosis model for predicting progression of HCM in a patient.

FIG. 14 is a flowchart of an example arrangement of operations for a computer-implemented method of predicting progression of HCM in a patient.

FIG. 15 is a flowchart of an example arrangement of operations for a computer-implemented method of training an ML medical prognosis model for predicting progression of HCM in a patient.

FIGS. 16A-C are charts showing example extracted clinical outcomes of a patient.

FIG. 17 is a schematic view of an example computing device that may be used to implement the systems and methods described herein.

### DETAILED DESCRIPTION

Patients with HCM often remain asymptomatic or mildly symptomatic. The prevalence of symptomatic hypertrophy is estimated in the U.S. at less than 1 in 3,000 adults. Patients are generally diagnosed at symptomatic stage after complaining of dyspnea, angina, fatigue, exercise intolerance, or presyncope and/or syncope on exertion. Detection of HCM in asymptomatic patients may occur during family screening relying on genetic testing or by an abnormal electrocardiogram obtained for other reasons (e.g., life insurance physical, non-cardiac surgery or procedure). Typically, the diagnosis is confirmed by echocardiogram and/or cardiac magnetic resonance imaging.

The New York Heart Association (NYHA) classification is often used in routine clinical practice and in trials to grade the extent of heart failure (HF) symptoms with exertion. HYHA classification classifies patients in one of four classes based on their limitations during physical activity. It varies from class I, corresponding to asymptomatic patients in ordinary physical activity, to class IV, corresponding to severe limitations where patients experience symptoms even while at rest.

Cardiac myosin inhibitors have recently emerged as a new treatment option for patients with HCM and are considered to have a disease-modifying potential. Trials for the effectiveness of cardiac myosin inhibitors have focused on symptomatic NYHA class II and class III HCM patients. As the cardiac myosin inhibitors treatment is disease specific, it can be expected to prevent asymptomatic patients from progression to symptomatic disease. However, it remains a challenge to test efficacy of cardiac myosin inhibitors for patients who are in an early stage of HCM, i.e., patients classified as asymptomatic NYHA class I or class II, or NYHA class II exhibiting only mild symptoms, because the progression of the disease can be slow, and, thus, measuring treatment effectiveness based on disease progression to a higher NYHA class may be impractical in a suitable time frame. Moreover, HCM is a heterogeneous disease that has a progression that is not currently known or well understood. Furthermore, the history of early stage HCM is not well documented or understood. Further still, few prognostic medical features have been identified for HCM, especially in the early stages of HCM. Therefore, there is a need for methods for prognosticating a progression of HCM for a patient. That is, methods for determining a medical prediction of a clinical outcome or course of HCM in a patient. As disclosed herein, predicting the clinical outcome or course may include medically predicting a probability of experiencing a particular progression of HCM by a particular threshold date (e.g., within the next 3 years). That is, for example, a disease progression from a first NYHA class assessment to a second NYHA class assessment higher than the first NYHA class assessment, or an occurrence of a cardiovascular event due to HCM. In some examples, the predicted outcomes for a plurality of patients is used to determine inclusion and/or exclusion criteria for selecting a pool of patients for clinical studies.

Implementations herein are directed toward training machine-learning (ML) medical prognosis models to teach the ML medical prognosis models to predict symptomatic progression and cardiovascular events for patients diagnosed with HCM. Implementations herein are also directed toward using such ML medical prognosis models to process medical data for a patient to infer or identify, for the patient, biomarkers of disease progression from asymptomatic or mildly symptomatic to a more severe stage or predict a likelihood of cardiovascular events. Notably, the medical data used to train the ML medical prognosis models or to predict HCM progression may be unstructured data (e.g., raw images or raw measurements). Advantageously, the ML medical prognosis models disclosed herein are interpretable such that they may, for example, be used to produce medical findings that are actionable by medical professions and/or to derive clinically-relevant criteria that may be used to direct care of patients diagnosed with HCM and/or to select patients for clinical trials.

Example medical data obtained for a patient may include, but is not limited to, clinical factors, medical imaging data such as echocardiograms (echo) images, cardiac measurements (e.g., left atrial area at end diastole, left ventricular area at end diastole, atrio-ventricular coupling, and left atrio-ventricular coupling index at end diastole), cardiovascular magnetic resonance (CMR) images, electrocardiogram (ECG) measurements, laboratory test results, genomics, and/or functional tests. The results output from these ML medical prognosis models may be used to identify patients at risk of disease progression together with insights on the size of the population at risk for assessing the feasibility of conducting a trial for the patients identified to be at risk. Specifically, the ML medical prognosis models may be trained and used during inference to identify HCM patients at an early stage, i.e., patients classified as asymptomatic NYHA class I and NYHA class II exhibiting only mild symptoms, who are at risk of quickly progressing to a higher NYHA class and, thus, may benefit from treatment using cardiac myosin inhibitors. Stated differently, the ML medical prognosis models may be trained to perform prognostic enrichment by identifying a subgroup of HCM patients classified as asymptomatic NYHA class I or NYHA class II exhibiting only mild symptoms who are likely to progress to a more severe stage. Prognostic enrichment refers to the selection of patients with a higher likelihood of having a NYHA class progression, or any other disease-related outcome of interest.

Example predicted cardiovascular events include, but are not limited to, cardiovascular hospitalizations, new diagnoses of atrial fibrillation (AF) hospitalized and non-hospitalized, episodes of heart failure (hospitalized and non-hospitalized requiring treatment), time to transition from non-obstructed HCM (nHCM) to obstructed HCM (oHCM), initiation and/or escalation of HCM therapies (e.g., beta blocker, Verapamil, Diltiazem, and/or disopyramide), interventional procedures (device implant, myectomy, alcohol septal ablation, heart transplant), episodes of lethal ventricular arrhythmia (VT/VF) leading to sudden cardiac arrest (resuscitated), hospitalization and/or appropriate implantable cardioverter defibrillator (ICD) shock, transient ischemic attack (TIA)+strokes, death (all cardiovascular causes, which includes sudden cardiac death), acute myocardial infarction (MI), worsening in exercise capacity (e.g., pVO2), and worsening in LVOT gradient.

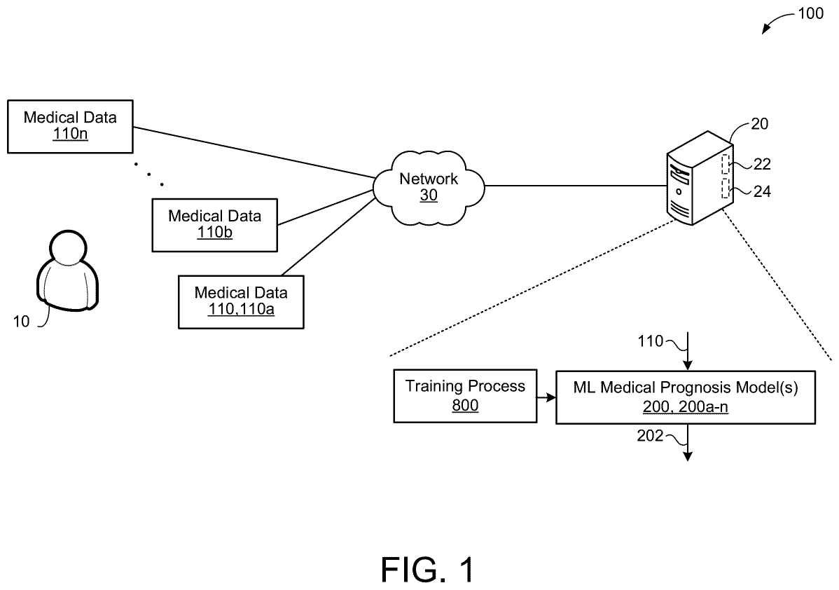

FIG. 1 is a diagram of an example of a system 100 for medically predicting a progression of hypertrophic cardiomyopathy (HCM) in a patient 10 using multidimensional medical data 110, 110a-n (also referred to herein a multi-modal medical data) collected for the patient 10 and spanning multiple different modalities (i.e., different tests or measurements). The predicted progression may include one or more of a predicted clinical outcome of, or course of, HCM in the patient 10 by a threshold date. The clinical outcome or course may be, for example, a progression of HCM from a first NYHA class assessment to a second NYHA class assessment higher than the first NYHA class assessment (e.g., from NYHA class I to NYHA class II, from NYHA class II to NYHA class III, or from non-obstructed HCM (nHCM) to obstructed HCM (oHCM)), or an occurrence of a cardiovascular event due to HCM by a particular threshold date. A cardiovascular event may be, for example, a cardiovascular-related hospitalization, a new diagnosis of atrial fibrillation (AF), an episode of heart failure (HF) requiring treatment, an episode of lethal ventricular arrhythmia leading to sudden cardiac arrest, an appropriate implantable cardioverter defibrillator (ICD) shock, a transient ischemic attack (TIA), a stroke, death, an acute myocardial infarction (MI), a worsening in venous oxygen partial pressure (pVO2), or a worsening in left ventricular outflow tract (LVOT) gradient. The multidimensional medical data 110 for the patient 10 may include, but is not limited to, clinical actors, medical imaging data such as echocardiograms (echo) images, cardiovascular magnetic resonance (CMR) images, cardiac measurements (e.g., left atrial area at end diastole, left ventricular area at end diastole, atrio-ventricular coupling, and left atrio-ventricular coupling index at end diastole), electrocardiogram (ECG) measurements, laboratory test results, genomics, and/or functional tests.

The system 100 includes a computing device 20 (e.g., a computer, a laptop, a tablet, a smartphone, a local server, a remote server, or a server of a distributed system executing in a cloud-computing environment, etc.) configured to process the multidimensional medical data 110 to medically predict a progression of HCM in the patient 10. Here, the multidimensional medical data 110 may be collected for the patient 10 during a threshold time period after an index date that corresponds to when the patient 10 was diagnosed with HCM. In some examples, the system 100 also receives the multidimensional medical data 110 as follow-up multidimensional medical data for the patient 10 that is collected for the patient 10 during a follow-up visit, extracts baseline features from the multidimensional medical data 110, and extracts follow-up features from the follow-up multidimensional medical data. In some examples, if a closest date for which measurements have been made or examinations have been performed is less than a threshold time from the patient's index date, then the measurement or examination performed may be used as baseline medical data. Otherwise, the measurement or exam may be considered missing. The threshold time may be, for example, 3 months. Here, processing the multidimensional medical data 110 to medically predict a progression of HCM in a patient 10 includes processing, using one or more trained ML medical prognosis models 200, the baseline features and the follow-up features. In some implementations, when it is determined that the predicted probability satisfies a threshold probability value, the patient 10 is selected for inclusion in a clinical trial or treatment of the patient 10 with a cardiac myosin inhibitor is initiated.

The computing device 20 includes data processing hardware 22 and memory hardware 24 in communication with the data processing hardware 22. The memory hardware 24 stores machine- or computer-readable instructions that, when executed by the data processing hardware 22, cause the data processing hardware 22 to perform operations disclosed herein for predicting a progression of HCM for the patient 10 or training an ML medical prognosis model 200 for predicting a progression of HCM for the patient 10. The computing device 20 may obtain the multidimensional medical data 110 from any number and/or type(s) of data stores (not shown for clarity of illustration) via any number and/or type(s) of public and/or private communication network(s) 30. Alternatively, the computing device 20 may be in direct communication with the data stores via any number and/or type(s) of wired or wireless digital communication interfaces (e.g., Bluetooth®, USB, etc.).

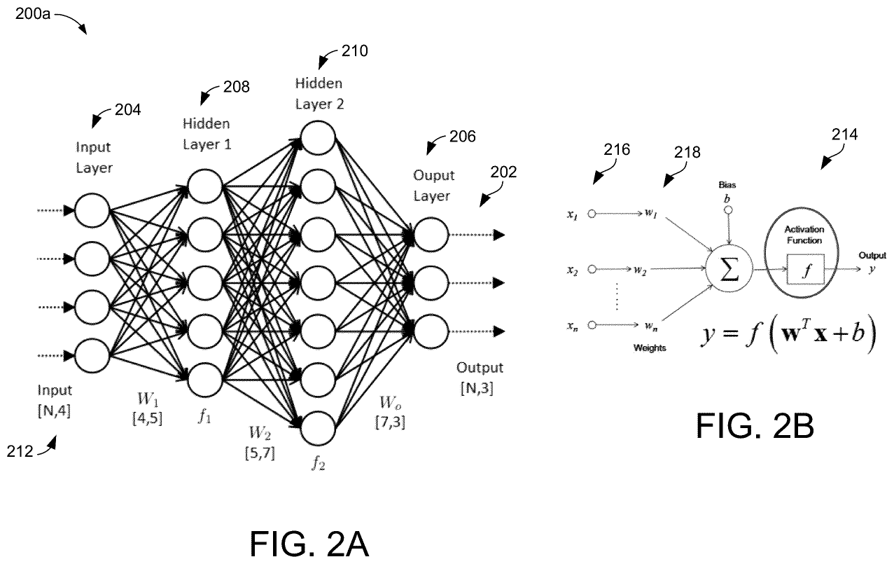

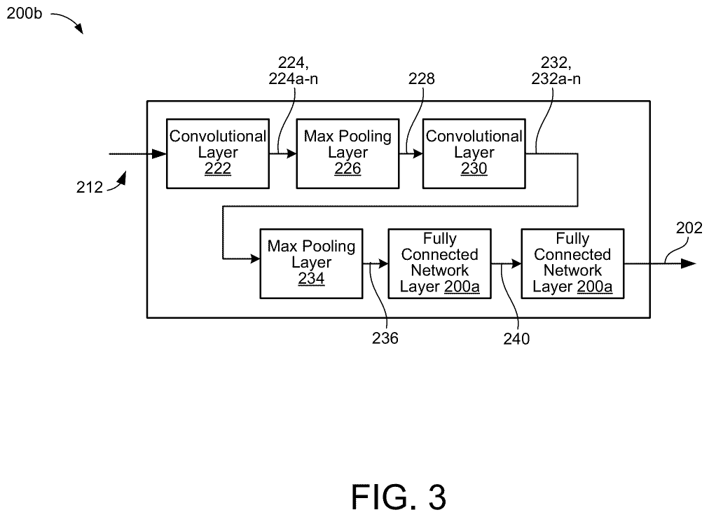

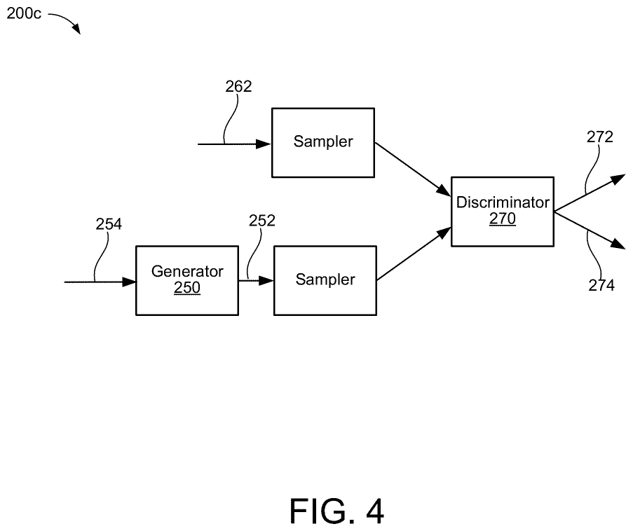

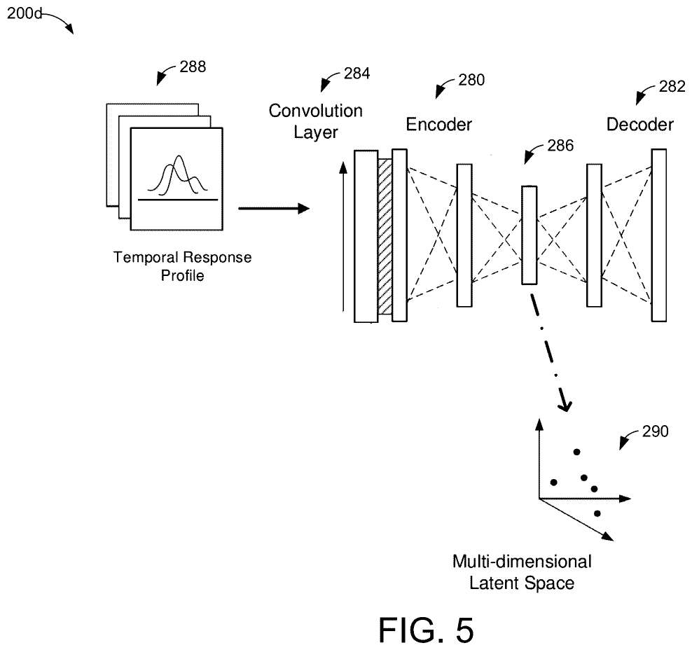

To medically predict a progression of HCM in the patient 10, the computing system 100 executes one or more trained ML medical prognosis models 200, 200a-n (also referred to herein as model(s) 200). The model(s) 200 may be trained using ML techniques. In some implementations, the model(s) 200 include one or more of a survival model, an artificial neural network (ANN) (see FIG. 2), a convolutional neural network (CNN) (see FIG. 3), an attention-based neural network, a generative neural network (GAN) including one or more conditions corresponding to modalities of multidimensional medical data (see FIG. 4), an autoencoder, a variational autoencoder (VAE) (see FIG. 5), a regression model (e.g., a multivariate survival model or a multivariate temporal response function (mTRF) model that includes independent variables corresponding to modalities of multidimensional medical data), a linear model, a non-linear model, a support vector machine, a decision tree model, a random forest model, an ensemble model, a Bayesian model, a naïve Bayes model, a k-means model, a k-nearest neighbors model, a principal component analysis, a Markov model, or any combinations thereof. The model(s) 200 may be, or include, software and/or machine- or computer-readable instructions stored on memory hardware (e.g., the memory hardware 24) that, when executed by a processing unit (e.g., the data processing hardware 22), cause the computing device 20 to predict a progression of HCM in the patient 10.

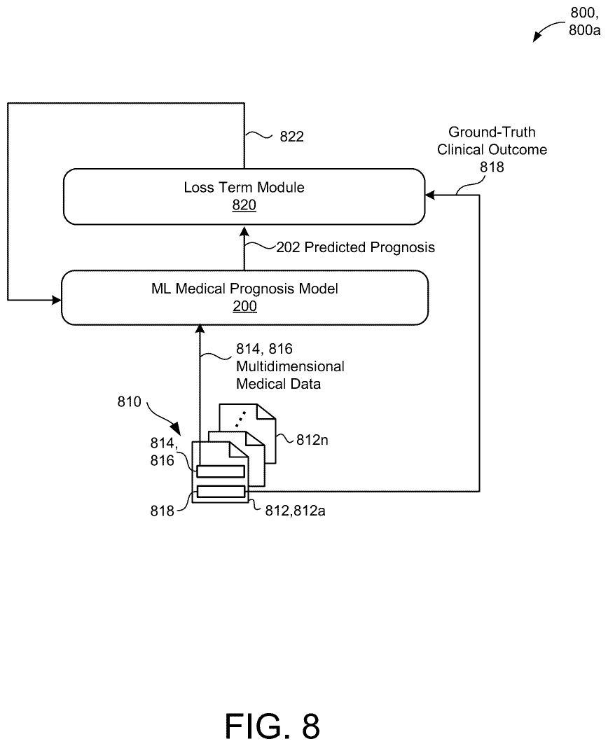

In some implementations, the model(s) 200 is trained using a training process 800 (e.g., see FIGS. 8-11) that obtains, for each patient of a plurality of patients, corresponding baseline training data including baseline multidimensional medical data 110 spanning multiple different modalities, and corresponding follow-up training data including follow-up multidimensional medical data 110 spanning multiple different modalities and collected after the baseline training data, and a corresponding ground-truth clinical outcome. The training process 800 trains the model(s) 200 on the baseline training data and the follow-up training data to teach the model(s) 200 to learn how to predict the corresponding ground-truth clinical outcomes.

FIGS. 2A and 2B are a schematic view of an example artificial neural network (ANN) model 200a for predicting progression of HCM in the patient 10. The ANN model 200a of FIG. 2 is fully connected, such that each neuron or node in one layer of the ANN model 200a connects to and has a feed-forward connection with every neuron in the next layer of the ANN model 200a. The connections in the ANN model 200a are unidirectional going in the direction from an input layer 204 to an output layer 206. The ANN model 200a has one input layer 204 and one output layer 206, with two or more hidden layers 208 and 210 between the input layer 204 and the output layer 206. Neural networks having more than one hidden layer are also called deep neural network.

Each neuron in the input layer 204 receives an input signal corresponding to an element in an input vector 212. In classifier neural networks, each neuron in the output layer 206 may represent a class. For example, if the ANN model 200a is configured to distinguish two disease outcome classes, e.g., disease vs. no disease, two output neurons of the output layer 206 may be used to represent the two disease outcomes. For another example, the ANN model 200a may be configured to output, e.g., 10 levels of severity of disease outcome. Here 10 output neurons may be used to represent the 10 levels of disease severity. In these implementations, the final outputs of the ANN model 200a are categorical. The activation functions for the output neurons of these categorial networks may be SoftMax functions in some implementations, resulting in a probability for each neuron, where all probabilities of the output neurons sum up to 1. Their loss functions may be cross entropy functions in some implementations. In other implementations, the ANN model 200a is designed to output a continuous variable. For example, it may be desirable to model a number of clinical events. In such implementations, the activation function of an output neuron may be a linear function, a sigmoid function, or another nonlinear function. The loss function may be, for example, a root mean square (RMS) error.

FIG. 2B shows the inputs and output of a neuron y 214 of the ANN model 200a. Here, the output y of the neuron 214 is determined by the input of its upstream neurons 216 weighted by the strength of the connections w 218 between neurons. The weighted sum of the inputs from the upstream neurons 216 is combined with a bias term b that ensures that the neuron 214 is activated to at least some degree. Then the combined weighted sum and bias b go through an activation function ƒ to provide the output of the neuron y 214.

FIG. 3 is a schematic view of an example convolutional neural network (CNN) 200b for predicting progression of HCM in the patient 10. Conventional ANNs, such as that shown in FIGS. 2A and 2B, may not be optimal for detecting spatial relations that are invariant across size, location, or orientation. CNNs may be better able capture such spatial relations and achieve size, location, and orientation invariance. The CNN model 200b may also be more effective than a conventional ANN in extracting features from temporal response profiles that have complex patterns and inter-profile relations in some implementations.

The CNN model 200b includes as an input layer 222 a first convolutional layer that uses a 5×5 kernel with one stride size 1 and n1 channels (for n1 feature maps). The input layer 222 extracts spatial features in each 5×5 kernel and generates a feature map 224, 224a-n for each channel. The CNN model 200b includes a first max-pooling layer 226 that uses a 2×2 kernel to downscale the feature maps 224 generated by the input layer 222. The CNN model 200b also includes a second convolutional layer 230 that uses a 5×5 kernel with one stride size and n2 channels (for n2 feature maps). The CNN model 200b also includes a second max-pooling layer 234 that uses a 2×2 kernel to downscale feature maps 232, 232a-n generated by the second convolutional layer 230. The further downscaled feature maps are then flattened to provide a vector 236, which becomes the input to a first fully connected layer 200a that may be analogous to the ANN model 200a of FIG. 2A. An output 240 of the first fully connected layer 200a becomes the input to a second fully connected layer 200a that may also analogous to the ANN model 200a.

FIG. 4 is a schematic view of an example generative adversarial network (GAN) model 200c for predicting progression of HCM in the patient 10. Here, the GAN model 200c may include one or more conditions corresponding to modalities of the multidimensional medical data 110. In some implementations, the GAN model 200c is configured to provide as output a clinical progression profile indicating disease severity at different times. In some implementations, the clinical progression profile values may be discretized to represent different disease states such as NYHA classes I-V. In some examples, a clinical progression profile is empirically obtained from repeatedly measuring at least one clinical outcome over a period of time. The clinical progression profile may then be used to train the model(s) 200.

The GAN model 200c includes a generator neural network 250 that generates data 252 mimicking real data such as images, data, or clinical progression profiles. The generator neural network 250 uses random noise as an input 254, which provides a stochastic mechanism for the generated data 252 so that the generated data 252 is analogous to, but not identical to, real data 262. The data 252 generated by the generator neural network 250 (e.g., generated images 252) and the real data 262 (e.g., real images 262) may be used as labeled data to train a discriminator neural network 270. The discriminator neural network 270 may improve its ability to discriminate the real images 262 and the generated images 252 through training, and its loss function 272 is configured to increase the discriminator's ability to discriminate between the real images 262 and the generated images 252. A complementary loss function 274 is used to train the generator neural network 250. As the generator neural network 250 is initially poor at generating images 252 similar to real images 262, the discriminator neural network 270 is penalized less and the generator neural network 250 is penalized more. As the generator neural network 250 gets better, the discriminator neural network 270 is penalized more and the generator neural network 250 is penalized less. The GAN model 200c typically stabilizes when the discriminator neural network 270 is able to, which may be varied depending on the scenario. In a non-limiting example, the GAN model 200c stabilizes when the discriminator neural network 270 is able to accurately discriminate about 50% of the time. In the GNN model 200c, each neuron in a final output layer may represent a value in the output data space (e.g., a pixel intensity in an image).

In some implementations, the GAN model 200c includes a conditional generative adversarial network (cGAN) model that enables the generator neural network 250 to generate images 252 that meet a condition, e.g., the gender of a patient in an image. In some implementations, such conditioning may be achieved by labeling training samples according to the conditions and encoding the conditions as one or more variables in the inputs to the generator neural network 250. For example, medical data may be categorized into different conditions and the training data may be also divided according to the different conditions. The cGAN model's input to the generator neural network 250 may include one or more features encoding the conditions of the training data. During data generation, by specifying input feature corresponding to a medical condition, the generator neural network 250 may generate temporal response profiles meeting the condition.

FIG. 5 is a schematic view of an example variational auto encoder (VAE) model 200d for predicting progression of HCM in the patient 10. The VAE model 200d is a neural network model that includes an encoder network 280 for encoding input into latent variables in a latent space 290, and a decoder network 282 for decoding and reconstructing data from the latent space 290. In some examples, the VAE model 200d is an unsupervised learning model that can extract features in reduced dimensions, as the VAE model 200d can be trained using unlabeled data by comparing the unencoded input and the decoded output to provide a loss function. The VAE model 200d may also be used to generate data that is analogous to, but different from, its training data. The difference may be derived from the stochastic sampling of the latent variables.

As illustrated, the VAE model 200d may optionally include a convolution layer 284 at an input side, the multilayer encoder portion 280, a hidden layer 286, and the multilayer decoder portion 282. The VAE model 200d is configured to receive input data 288 such as temporal response profiles acquired from a test sample. In some implementations, a temporal response profile is implemented as a clinical progression profile. In some implementations, unimodal or multi-modal medical data is provided as input 288 to the VAE model 200d. Optionally, the input data 288 is organized and provided to the convolution layer 284, which is configured to extract potentially relevant features from the data. The VAE model 200d is configured such that the input data 288 filtered by the convolution layer 284 is processed by the encoder layer 280 and decoded by the decoder layer 282. Between the encoder 280 and decoder 282 is the hidden layer 286 that is configured to hold the fully encoded data in the latent space 290.

In the example shown, the hidden layer 286 holds a multidimensional latent space representation 290 of the fully encoded data. The latent space representation 290 includes multiple data points, each associated with a particular sample or a particular reading taken from a sample. In a conventional autoencoder, data in the hidden layer is static. However, in the VAE model 200d, data in the hidden layer 286 is associated with random noise sampled from a distribution such as a Gaussian distribution, providing stochastic variation for the VAE model 200d. As such, encoded and decoded data may be different, allowing generation of new data that is analogous to, but different from, training data. Therefore, VAEs may be used as generative models in some implementations.

Each data point in the latent space 290 includes a feature vector, which has fewer dimensions than input and output data of the VAE model 200d. Therefore, VAEs may also be used to extract features, reduce dimensions, and/or reduce noise. The extracted features with lower dimensions may be used as input to other ML models such as an ANN or regression model.

Training of the VAE model 200d may employ loss functions and/or other techniques that project input data into a latent space in a probabilistic manner. In some implementations, the loss functions employ a regularization term utilizing a Kullback-Leibler divergence. The feature extractor projects data not as discrete values but as distributions of values on axes in the latent space 290. The distributions may be characterized by, e.g., their central tendencies (means, medians, etc.) and/or their variances in the latent space 290. The training may encourage the learned distribution (in latent space 290) to be similar to the true prior distribution (the input data).

In some implementations, the model(s) 200 include one or more survival models. A survival model is a type of model that analyzes time-to-event data. The primary focus of a survival model is on predicting the time until an event of interest occurs. The event in a survival model is often an endpoint like death, disease progression, disease recurrence, or failure of a component. However, the event may be any event whose timing is of interest. A unique aspect of survival models are survival functions that handle censored data. Censoring occurs when the event has not happened for some subjects during the study period or when the subject is lost to follow-up. The exact time of the event for these subjects is unknown, which is a significant challenge in the analysis. A survival function of a survival model represents the probability of surviving (or the probability of the event not occurring) up to a certain time. Survival functions are key outputs of survival models. In some implementations, a survival model provides an output of a probability of progression of a disease from one stage to another. A survival model's hazard function describes the instantaneous risk of the event occurring at a given time, assuming it hasn't occurred yet. The survival model's hazard function is useful for understanding how risk factors affect the likelihood of the event over time.

In some implementations, a survival model includes a Kaplan-Meier estimator model, which is a non-parametric statistic used to estimate the survival function from lifetime data. A Kaplan-Meier model is employed to construct survival curves for patient cohorts categorized based on a combination of genetic, clinical, and echocardiographic markers. This non-parametric estimator is suitable for determining the probability of HCM progression over time within cohorts. The survival curves generated provide a visual representation of the time-to-event data, illustrating the likelihood of disease progression or the occurrence of significant cardiac events over specified intervals. The Kaplan-Meier estimator is particularly adept at handling censored data, a common challenge in medical studies where patients might be lost to follow-up, or the event of interest has not occurred by the study's end. Additionally, or alternatively, a survival model may include a Cox proportional hazards model, which is a semi-parametric model widely used in medical research. A Cox proportional hazards model models the hazard rate as a function of several covariates. Additionally, or alternatively, a survival model may include a parametric survival model, which includes exponential, Weibull, log-normal models, etc. features, and assumes a specific distribution for the survival times.

In some implementations, the model(s) 200 include one or more multivariate temporal response function (mTRF) models. Here, an mTRF model may include independent variables corresponding to modalities of multidimensional medical data. An mTRF model may be configured to provide as output a clinical progression profile, where temporal response function (TRF) values correspond to disease severity at different times. In some examples, the TRF values are discretized to represent disease states such as NYHA classes I-V. In some implementations, an mTRF model is used to predict a disease progression profile. An mTRF model may be trained to receives as input multidimensional medical data 110 of the patient 10 and predict a clinical progression profile for the patient 10, where TRY function values correspond to disease severity at different times.

An TRF is a univariate regression model, in which n temporal response functions are recorded by N channels, the N channels representing time points of interest. The TRF assumes that an instantaneous response r(t, n) sampled at times t=1,2, . . . T at channel n is provided by a convolution of a stimulus property s(t) with an unknown channel-specific TRF w(τ, n). The response can be represented in discrete time as:

$$r ( t , n ) = {\sum }_{τ} w ( τ , n ) s ( t - τ ) + ε ( t , n ) \qquad EQN ( 1 )$$

where ε(t, n) is the residual response at each channel n not explained by the response. A TRF can be thought of as a filter that describes the linear transformation of an ongoing stimulus (e.g., a medical data variable) to the ongoing channel response. The TRF w(τ, n) describes this transformation from stimulus s to response r for a specified range of time lags τ relative to the instantaneous occurrence of the stimulus feature s(t). The range of time lags τ over which to calculate w(τ, n) might be that typically used to capture a patient response to a medical variable, such as a range determined by empirical medical observation. The TRF w(τ, n) may estimated by, for example, minimizing the mean-squared error (MSE) between the actual temporal response profile r(t, n) and that predicted by the convolution \hat{r}(t, n) as:

$$min ε ( t , n ) = \frac{1}{τ} {{\sum }_{τ} [ r ( t , n ) - \hat{r} ( t , n ) ]}^{2} \qquad EQN ( 2 )$$

Here, the univariate TRF receives a single stimulus variable and provides a clinical progression profile in each channel of N channels. If multiple stimuli are encoded as multiple independent variables, an mTRF model can be applied.

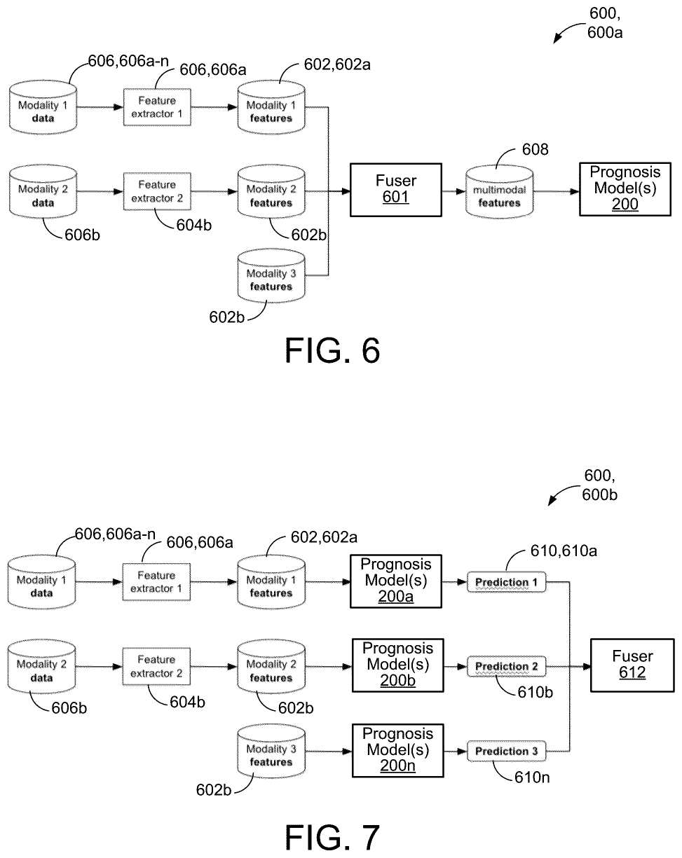

FIG. 6 is a schematic view of an example fusion architecture 600, 600a that includes a fuser 601 that fuses features 602, 602a-n extracted by a plurality of different feature extractors 604, 604a-n corresponding to different data modalities 606, 606a-n into a set of fused multimodal features 608. Here, the set of fused multimodal features 608 are processed by the model(s) 200 for predicting progression of HCM in the patient 10. In some examples, the fuser 601 fuses the features 602 using a weighted sum (e.g., a linear sum) of the features 602.

FIG. 7 is a schematic view of another example fusion architecture 600, 600b. In the illustrated example of FIG. 7, each extracted feature 602 is processed by a corresponding model 200, and then predictions 610, 610a-n generated by the models 200 are fused by a fuser 612. In some examples, the fuser 612 fuses the predictions 610 using a weighted sum (e.g., a linear sum) of the predictions 610.

The model(s) 200 may be training using any number and/or type(s) of training methods and processes, such as unsupervised, self-supervised, semi-supervised, and supervised training methods. In certain embodiments, a model 200, such as a VAE, is trained in a semi-supervised fashion that employs both labeled and unlabeled training data. Examples of semi-supervised training techniques are described in a paper entitled “A Survey on Deep Semi-supervised Learning” by Yang, X., Song, Z., King, I., & Xu, Z. (2021), http://arxiv.org/abs/2103.00550, which is incorporated herein by reference in its entirety. In some implementations, a model 200 is trained in one or more iterations, and in fact may employ multiple separate models 200, some serving as a basis for transfer learning of later developed refinements or versions of the model. In some implementations, a feature extractor is partially trained using supervised learning and partially trained using unsupervised learning.

In some implementations, techniques such as model validation, cross-validation, and bootstrap methods are employed to assess the reliability of the predictions and to guard against overfitting, a common concern in predictive modeling. Additionally, or alternatively, learning may be conducted in multiple stages using multiple training data sources using a mechanism such as transfer learning. Transfer learning is a training process that starts with a previously trained model and adopts that model's architecture and current parameter values (e.g., previously trained weights and biases) but then changes the model's parameter values to reflect new or different training data. In various embodiments, the original model's architecture, including convolutional windows, if any, and optionally its hyperparameters, remain fixed through the process of further training such as via transfer learning. In some examples, one or more training routines produce a first trained preliminary model 200. Once fully trained with training data, the preliminary model 200 may be used as a starting point for, e.g., training a second model 200. The training of the second model 200 may start by using a model having the architecture and parameter settings of the first trained model 200 but refines the parameter settings by incorporating information from additional training data.

Additionally, or alternatively, training of the model(s) 200 may occur in two stages. During the first stage, the model(s) 200 may be trained to predict a risk of class progression (or other endpoint of interest) using survival models. Example survival models may include cox regression models, tree-based models, and deep neural network-based using different modalities. A nested cross validation (NCV) algorithm may then be applied to select the best model(s) 200 based on both discriminative and calibration performance. During a second stage, a calibration step may be applied in order to transform an output of a model 200 into an interpretable outcome, such as a probability of experiencing a progression in NYHA class in three years.

FIG. 8 is a schematic view of an example training process 800, 800a for training a model 200 for predicting progression of HCM in the patient 10. The training process 800a may execute on the computing device 20 (i.e., on the data processing hardware 22) or on the data processing hardware of another computing system, such as a remote physical or virtual server. In the example shown, the training process 800a trains the model 200 using a training data set 810 that includes a plurality of training samples 812, 812a-n. Here, each particular training sample 812 of the plurality of training samples 812 includes corresponding baseline training data 814 including baseline multidimensional medical data 110 spanning multiple different modalities, corresponding follow-up training data 816 including follow-up multidimensional medical data 110 spanning multiple different modalities and collected after the baseline training data and a corresponding ground-truth clinical outcome 818.

For each particular training sample 812 in the training data set 810, the training process 800a processes, using the model 200, the corresponding baseline training data 814 and the follow-up multidimensional medical data 816 to generate a predicted prognosis 202, such as a probability of experiencing a progression of hypertrophic cardiomyopathy (HCM) of the patient by a threshold date. A loss term module 820 determines a loss 822 based on the corresponding ground-truth clinical outcome 818 and the predicted prognosis 202.

Thereafter, the training process 800a trains the model 200 based on the losses 822 to teach the ML medical prognosis model 200 to learn how to predict the corresponding ground-truth clinical outcomes 818. In some examples, the training process 800a trains the model 200 by adjusting, adapting, updating, fine-tuning, etc. one or more parameters or weights of the model 200 based on the losses 822.

In some examples, the training data set 810 is hosted on a site of an owner of the multidimensional medical data 110 of the training data set 810, which may include a hospital, educational institution, or other source. Without exposing the multidimensional medical data 110, the training data set 810 may be accessed via federated data access, and a worker node (not shown for clarity of illustration) on-site of the owner of the training data set 810 may train the model 200 on the respective the training data set 810. In this fashion, the model 200 may be trained by worker nodes associated with different sources of the training data 810 so that the training data 810 is never shared or interpretable, only the model 200 is trained during multiple loops each corresponding to a different worker node. Federated data access and federated learning techniques for training the prognosis models are described in PCT Patent Application No. PCT/US2021/061417, filed on Dec. 1, 2021, which claims priority to European Patent Application No. 20306478.7, filed on Dec. 1, 2020. The disclosures of these prior applications are considered part of the disclosure of this application and are hereby incorporated by reference in their entireties.

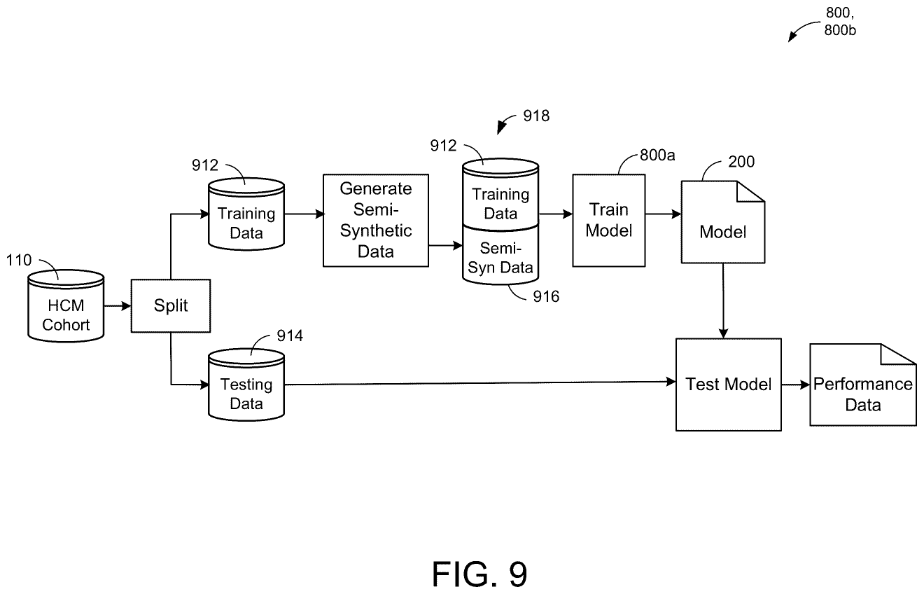

FIG. 9 is a schematic view of another example training process 800, 800b for training a model 200 for predicting progression of HCM in the patient 10. The training process 800b may execute on the computing device 20 (i.e., on the data processing hardware 22) or on the data processing hardware of another computing system, such as a remote physical or virtual server. The training process 800b splits multidimensional medical data 110 for a plurality of patients 10 into a training data set 912 and a testing data set 914. In some examples, the training data set 912 is augmented with semi-synthetic training data 916 to form training data 918. The training data 918 is then used, for example, using the training process 800a of FIG. 8 to train an ML medical prognosis model 200. Thereafter, the training process 800b tests the performance of the trained ML medical prognosis model 200 using the testing data set 914. In some examples, the training process 800b trains the model 200 by adjusting, adapting, updating, fine-tuning, etc. one or more parameters or weights of the model 200 based on computed losses.

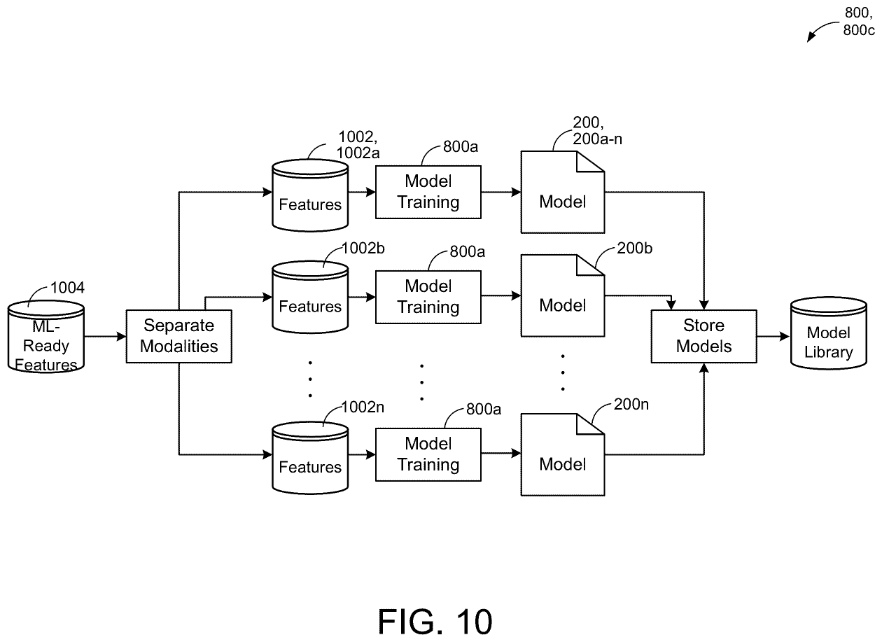

FIG. 10 is a schematic view of another example training process 800, 800c for training a plurality of models 200 for predicting progression of HCM in the patient 10. The training process 800c may execute on the computing device 20 (i.e., on the data processing hardware 22) or on the data processing hardware of another computing system, such as a remote physical or virtual server. In the illustrated example of FIG. 10, the training process 800a of FIG. 8 is repeated for each modality 1002, 1002a-n of a training data set of ML-ready features 1004 to train a corresponding model 200 for each modality 1002. In some examples, the training process 800c trains the models 200 by adjusting, adapting, updating, fine-tuning, etc. one or more parameters or weights of the models 200 based on computed losses.

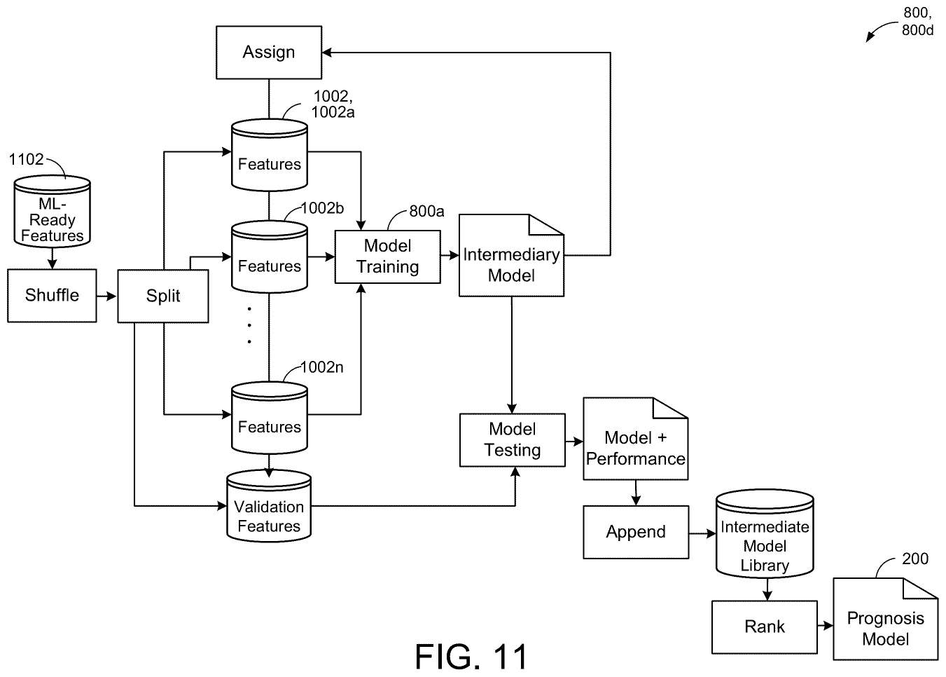

FIG. 11 is a schematic view of yet another example training process 800, 800d for training a model 200 for predicting progression of HCM in the patient 10. The training process 800d may execute on the computing device 20 (i.e., on the data processing hardware 22) or on the data processing hardware of another computing system, such as a remote physical or virtual server. In the illustrated example of FIG. 11, the ML medical prognosis model 200 is trained using nested cross-validation, with an intermediary model 1104 trained using the training process 800a of FIG. 8. In some examples, the training process 800d trains the models 200 by adjusting, adapting, updating, fine-tuning, etc. one or more parameters or weights of the model 200 based on computed losses.

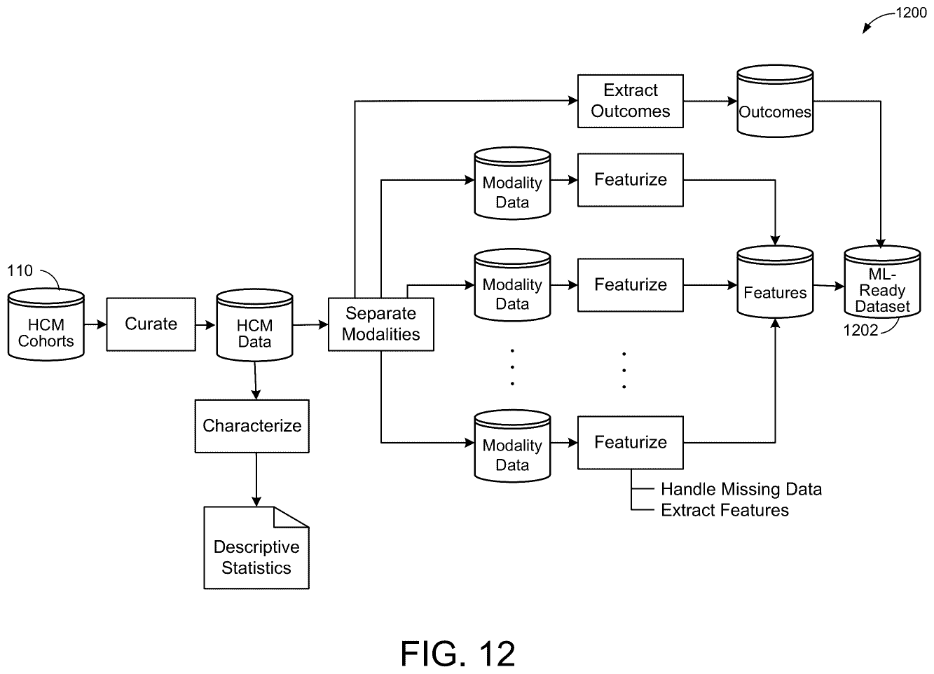

FIG. 12 is a schematic view of an example data pre-processing process 1200 for generating a training data set of ML-ready features 1202 that may be used as the training data set 810 of FIG. 8, the data 912 and 914 of FIG. 9, the training data set of ML-ready features 1004 of FIG. 10, and/or the training data set of ML-ready features 1102 of FIG. 11. The process 1200 splits the multidimensional medical data 110 based on modality, extracts features for each modality, extracts clinical outcomes from clinical data, and stores the extracted features in the training data set of ML-ready features 1202.

In the illustrated example of FIG. 12, a patient 10 may be selected for inclusion in the multidimensional medical training data 110 based on one or more inclusion criteria and/or exclusion criteria. Example inclusion criteria include, but are not limited to:
Adult patients with a confirmed diagnosis of HCM (e.g., echo-based or CMR evidence of LVH not due to other causes)
Patients who are NYHA class I or “early stage” NYHA class II at baseline and who have at least one follow-up that includes their NYHA class (which may be NYHA class I or higher according to AHA/ACC criteria). Here, “early stage” NYHA class II defined as NYHA Class II patients with mild symptoms potentially treated with at most one background medication (beta blocker or calcium channel blocker disopyramide), and
   No history of atrial fibrillation, and
   Resting or provoked LVOT<50 mmHg, and
   NT-pro-BNP<300 pg/m1, and
   LAVi<35 m1/m2 (if available), and
   E/e′<14 (if available)
Patients must have data (clinical, imaging, biological, omics, and/or exercise testing) available at baseline and longitudinally
Patients addressed in the primary or secondary cardiology department
Follow-up for at least 3 years Example inclusion criteria include, but are not limited to, patients having non-sarcomeric HCM (e.g., any phenocopy disease) such as Amyloidosis, Hemochromatosis, Fabry disease, Aortic valve stenosis, or Left ventricular Hypertrophy (LVH) in athletic heart syndrome, or hypertensive disease.

During a patient's follow-ups, his or her NYHA class can be assessed several times resulting in a time series of class assessment for use in deriving a time of progression that may be used to train the model(s) 200. To ease readability, the case of progression from class I to class II may be assessed, however, the same techniques are equally applicable for the cases of progression from “early stage” NYHA II to higher NYHA class.

Here, time-to-progression may be defined as a time between the baseline (index date) and a first time that the progression is observed in the patient follow-up. If no progression occurs during the follow-up, the patient may be considered as censored. The time of censoring will be set to the time of the last visit of the patient. To increase a size of the training set, data collected from patient follow-ups may be used to generate synthetic patients. For instance, each time a patient has a follow-up after the index date and there is no progression in class, each of these follow-up dates may be labeled as a synthetic observation for use as a new index date for a synthetic patient.

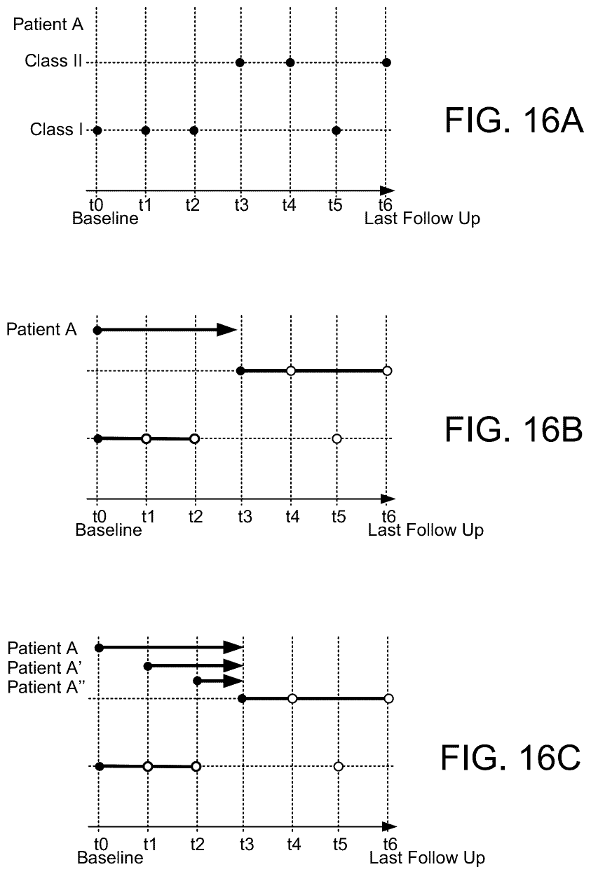

In some instances, a patient's HCM may be assessed as moving back and forth between two NYHA classes, as shown in FIG. 16A. In such examples, the patient 10 may, as shown in FIG. 16B, be considered to have reached the higher of the two NYHA classes the first time they are assessed at the higher NYHA class. Additionally, or alternatively, as shown in FIG. 16C, the patient's multidimensional data 110 may be used to create one or more synthetic patients corresponding to the various times the patient 10 was classed at the higher NYHA class. Such synthetic patients may be used as additional training samples for training the model(s) 200.

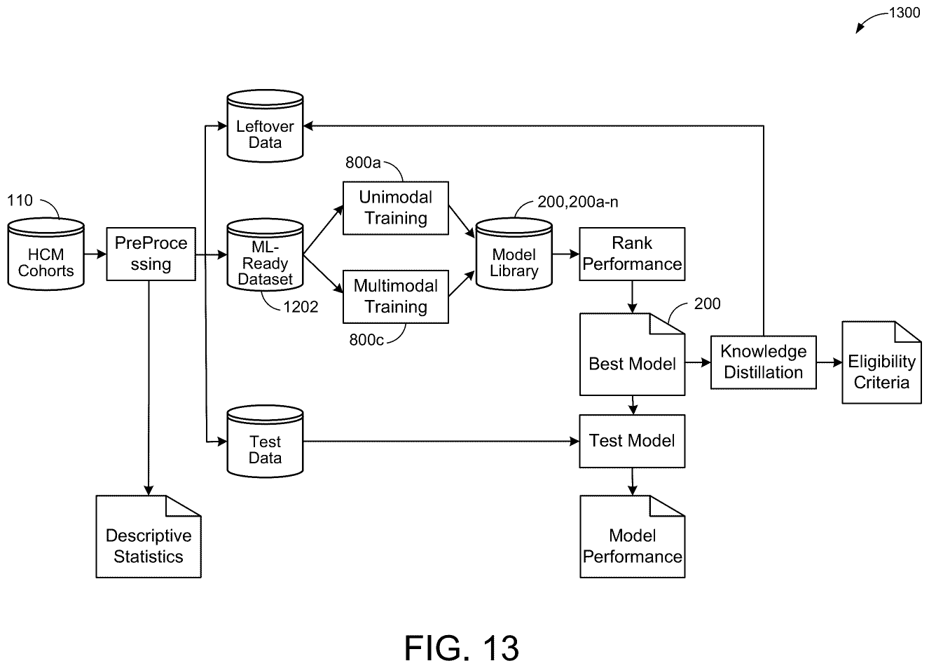

FIG. 13 is a schematic view of an example process 1300 for generating and selecting an ML medical prognosis model 200 for predicting progression of HCM in the patient 10.

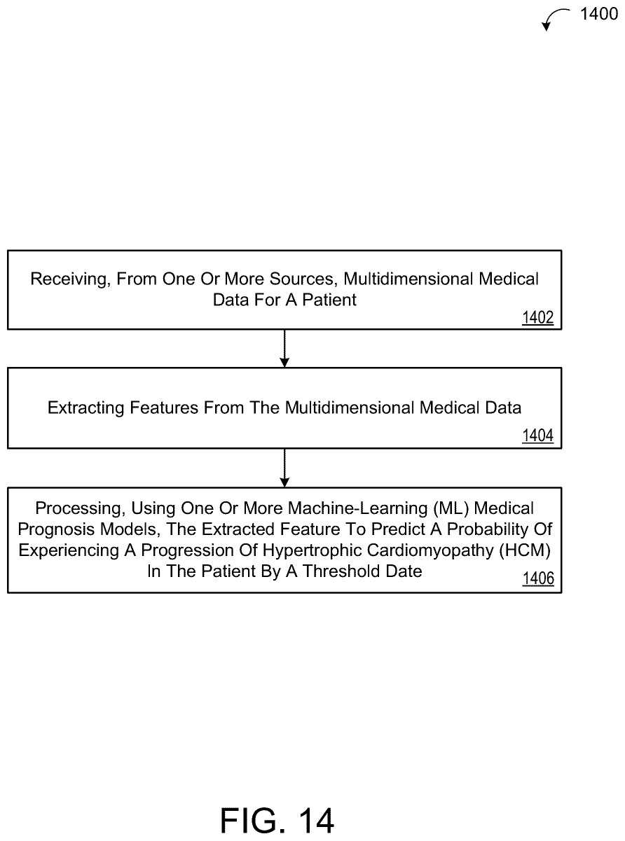

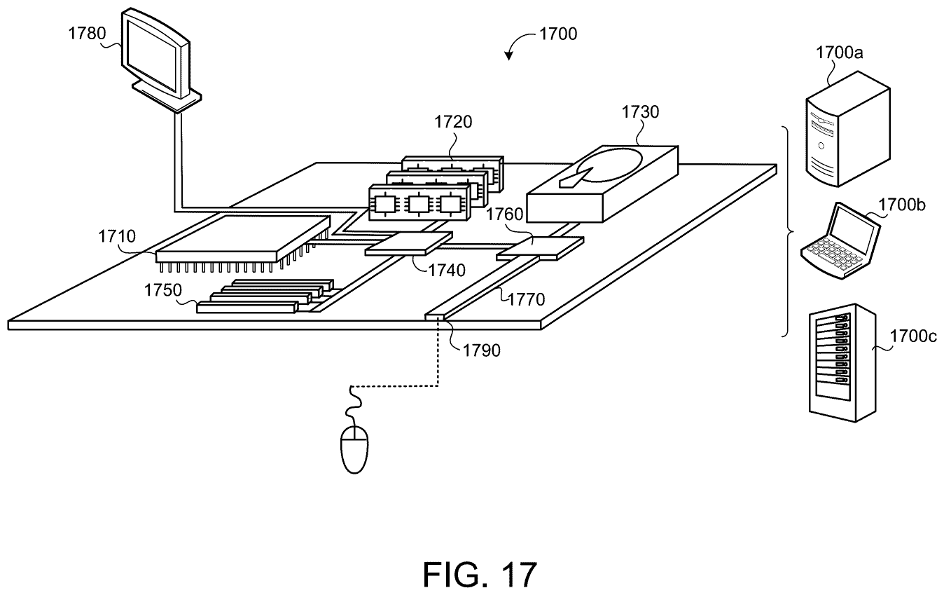

FIG. 14 is a flowchart of an example arrangement of operations for a computer-implemented method 1400 of predicting progression of HCM in the patient 10. The operations may be performed by data processing hardware 1710 (FIG. 17) (e.g., the data processing hardware 22 of the computing device 20) based on executing instructions stored on memory hardware 1720 (e.g., the memory hardware 24 of the computing device 20). Many other ways of implementing the method 1400 may be employed. For example, the order of execution of the operations may be changed, and/or one or more of the operations and/or interactions may be changed, eliminated, sub-divided, or combined. Additionally, the operations of FIG. 14 may be carried out sequentially and/or in parallel by, for example, separate processing threads, processors, devices, discrete logic, circuits, etc.

At operation 1402, the method 1400 includes receiving, from one or more sources, multidimensional medical data 110 for the patient 10. At operation 1404, the method 1400 includes extracting baseline features from the multidimensional medical data 110. At operation 1406, the method 1400 includes processing, using one or more ML medical prognosis models 200, the extracted features to predict a probability of experiencing a progression of HCM in the patient 10 by a threshold date. Here, the one or more ML medical prognosis models 200 are trained using a training process 800 that includes obtaining, for each of a plurality of patients 10, corresponding baseline training data including baseline multidimensional medical data 110 spanning multiple different modalities, and obtaining, for each of the plurality of patients 10, corresponding follow-up training data including follow-up multidimensional medical data 110 spanning multiple different modalities and collected after the baseline training data, and a corresponding ground-truth clinical outcome.

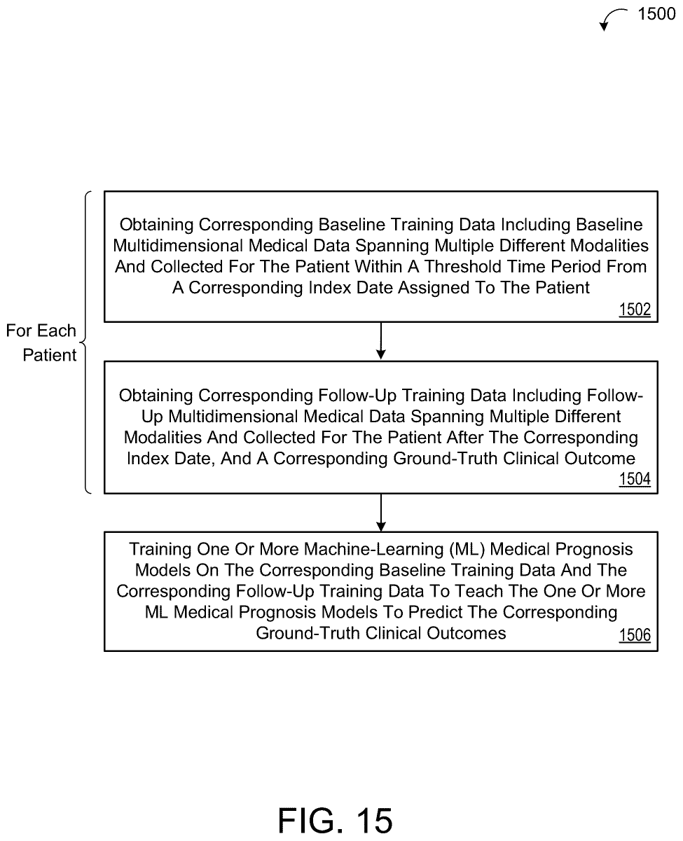

FIG. 15 is a flowchart of an example arrangement of operations for a computer-implemented method 1500 of training an ML medical prognosis model for predicting progression of HCM in the patient 10. The operations may be performed by data processing hardware 1710 (FIG. 17) (e.g., the data processing hardware 22 of the computing device 20) based on executing instructions stored on memory hardware 1720 (e.g., the memory hardware 24 of the computing device 20). Many other ways of implementing the method 1500 may be employed. For example, the order of execution of the operations may be changed, and/or one or more of the operations and/or interactions may be changed, eliminated, sub-divided, or combined. Additionally, the operations of FIG. 15 may be carried out sequentially and/or in parallel by, for example, separate processing threads, processors, devices, discrete logic, circuits, etc.

At operation 1502, the method 1500 includes, for each particular patient 10 of a plurality of patients 10 diagnosed with HCM and satisfying an inclusion criterion, obtaining corresponding baseline training data including baseline multidimensional medical data 110 spanning multiple different modalities and collected for the particular patient 10 within a threshold time period from a corresponding index date assigned to the particular patient 10. At operation 1504, the method 1500 includes, for each particular patient 10 of a plurality of patients 10 diagnosed with HCM and satisfying an inclusion criterion, obtaining corresponding follow-up training data including follow-up multidimensional medical data 110 spanning multiple different modalities and collected for the particular patient 10 after the corresponding index date, and a corresponding ground-truth clinical outcome. At operation 1506, the method 1500 includes training one or more ML medical prognosis models 200 on the corresponding baseline training data and the corresponding follow-up training data to teach the one or more ML medical prognosis models 200 to predict the corresponding ground-truth clinical outcomes.

FIG. 17 is a schematic view of an example computing device 1700 that may be used to implement the systems and methods described in this document. The computing device 1700 is intended to represent various forms of digital computers, such as laptops, desktops, workstations, personal digital assistants, servers, blade servers, mainframes, and other appropriate computers. The components shown here, their connections and relationships, and their functions, are meant to be exemplary only, and are not meant to limit implementations of the inventions described and/or claimed in this document.

The computing device 1700 includes a processor 1710 (i.e., data processing hardware) that can be used to implement the data processing hardware 22, memory 1720 (i.e., memory hardware) that can be used to implement the memory hardware 24, a storage device 1730 (i.e., memory hardware) that can be used to implement the memory hardware 24 or store models 200, ML-ready features, and training data, a high-speed interface/controller 1740 connecting to the memory 1720 and high-speed expansion ports 1750, and a low speed interface/controller 1760 connecting to a low speed bus 1770 and a storage device 1730. Each of the components 1710, 1720, 1730, 1740, 1750, and 1760, are interconnected using various busses, and may be mounted on a common motherboard or in other manners as appropriate. The processor 1710 can process instructions for execution within the computing device 1700, including instructions stored in the memory 1720 or on the storage device 1730 to display graphical information for a graphical user interface (GUI) on an external input/output device, such as display 1780 coupled to high-speed interface 1740. In other implementations, multiple processors and/or multiple buses may be used, as appropriate, along with multiple memories and types of memory. Also, multiple computing devices 1700 may be connected, with each device providing portions of the necessary operations (e.g., as a server bank, a group of blade servers, or a multi-processor system).

The memory 1720 stores information non-transitorily within the computing device 1700. The memory 1720 may be a computer-readable medium, a volatile memory unit(s), or non-volatile memory unit(s). The non-transitory memory 1720 may be physical devices used to store programs (e.g., sequences of instructions) or data (e.g., program state information) on a temporary or permanent basis for use by the computing device 1700. Examples of non-volatile memory include, but are not limited to, flash memory and read-only memory (ROM)/programmable read-only memory (PROM)/erasable programmable read-only memory (EPROM)/electronically erasable programmable read-only memory (EEPROM) (e.g., typically used for firmware, such as boot programs). Examples of volatile memory include, but are not limited to, random access memory (RAM), dynamic random-access memory (DRAM), static random-access memory (SRAM), phase change memory (PCM) as well as disks or tapes.

The storage device 1730 is capable of providing mass storage for the computing device 1700. In some implementations, the storage device 1730 is a computer-readable medium. In various different implementations, the storage device 1730 may be a floppy disk device, a hard disk device, an optical disk device, or a tape device, a flash memory or other similar solid state memory device, or an array of devices, including devices in a storage area network or other configurations. In additional implementations, a computer program product is tangibly embodied in an information carrier. The computer program product contains instructions that, when executed, perform one or more methods, such as those described above. The information carrier is a computer- or machine-readable medium, such as the memory 1720, the storage device 1730, or memory on processor 1710.

The high-speed controller 1740 manages bandwidth-intensive operations for the computing device 1700, while the low-speed controller 1760 manages lower bandwidth-intensive operations. Such allocation of duties is exemplary only. In some implementations, the high-speed controller 1740 is coupled to the memory 1720, the display 1780 (e.g., through a graphics processor or accelerator), and to the high-speed expansion ports 1750, which may accept various expansion cards (not shown). In some implementations, the low-speed controller 1760 is coupled to the storage device 1730 and a low-speed expansion port 1790. The low-speed expansion port 1790, which may include various communication ports (e.g., USB, Bluetooth, Ethernet, wireless Ethernet), may be coupled to one or more input/output devices, such as a keyboard, a pointing device, a scanner, or a networking device such as a switch or router, e.g., through a network adapter.

The computing device 1700 may be implemented in a number of different forms, as shown in the figure. For example, it may be implemented as a standard server 1700a or multiple times in a group of such servers 1700a, as a laptop computer 1700b, or as part of a rack server system 1700c.

Various implementations of the systems and techniques described herein can be realized in digital electronic and/or optical circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

A software application (i.e., a software resource) may refer to computer software that causes a computing device to perform a task. In some examples, a software application may be referred to as an “application,” an “app,” or a “program.” Example applications include, but are not limited to, system diagnostic applications, system management applications, system maintenance applications, word processing applications, spreadsheet applications, messaging applications, media streaming applications, social networking applications, and gaming applications.

These computer programs (also known as programs, software, software applications, or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, non-transitory computer readable medium, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

The processes and logic flows described in this specification can be performed by one or more programmable processors, also referred to as data processing hardware, executing one or more computer programs to perform functions by operating on input data and generating output. The processes and logic flows can also be performed by special purpose logic circuitry, e.g., an FPGA (field programmable gate array) or an ASIC (application specific integrated circuit). Processors suitable for the execution of a computer program include, by way of example, both general and special purpose microprocessors, and any one or more processors of any kind of digital computer. Generally, a processor will receive instructions and data from a read only memory or a random-access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, one or more aspects of the disclosure can be implemented on a computer having a display device, e.g., a CRT (cathode ray tube), LCD (liquid crystal display) monitor, or touch screen for displaying information to the user and optionally a keyboard and a pointing device, e.g., a mouse or a trackball, by which the user can provide input to the computer. Other kinds of devices can be used to provide interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Unless expressly stated to the contrary, the phrase “at least one of A, B, or C” is intended to refer to any combination or subset of A, B, C such as: (1) at least one A alone; (2) at least one B alone; (3) at least one C alone; (4) at least one A with at least one B; (5) at least one A with at least one C; (6) at least one B with at least C; and (7) at least one A with at least one B and at least one C. Moreover, unless expressly stated to the contrary, the phrase “at least one of A, B, and C” is intended to refer to any combination or subset of A, B, C such as: (1) at least one A alone; (2) at least one B alone; (3) at least one C alone; (4) at least one A with at least one B; (5) at least one A with at least one C; (6) at least one B with at least one C; and (7) at least one A with at least one B and at least one C. Furthermore, unless expressly stated to the contrary, “A or B” is intended to refer to any combination of A and B, such as: (1) A alone; (2) B alone; and (3) A and B.

A number of implementations have been described. Nevertheless, it will be understood that various modifications may be made without departing from the spirit and scope of the disclosure. Accordingly, other implementations are within the scope of the following claims.

What is claimed is:

1. A computer-implemented method executed on data processing hardware that causes the data processing hardware to perform operations comprising:
receiving, from one or more sources, multidimensional medical data for a patient;
extracting features from the multidimensional medical data; and
processing, using one or more machine-learning (ML) medical prognosis models, the extracted features to predict a probability of experiencing a progression of hypertrophic cardiomyopathy (HCM) in the patient by a threshold date, wherein a training process trains the one or more ML medical prognosis models by:
obtaining, for each of a plurality of patients, corresponding baseline training data comprising baseline multidimensional medical data spanning multiple different modalities;
obtaining, for each of the plurality of patients, corresponding follow-up training data comprising:
follow-up multidimensional medical data spanning multiple different modalities and collected after the baseline training data; and
a corresponding ground-truth clinical outcome; and
training the one or more ML medical prognosis models on the baseline training data and the follow-up training data to teach the one or more ML medical prognosis models to learn how to predict the corresponding ground-truth clinical outcomes.

2. The computer-implemented method of claim 1, wherein the one or more ML medical prognosis models comprise at least one of: a survival model, a neural network, a convolutional neural network (CNN), an attention-based neural network, a generative neural network, an autoencoder, a variational autoencoder (VAE), a regression model, a linear model, a non-linear model, a support vector machine, a decision tree model, a random forest model, an ensemble model, a Bayesian model, a naïve Bayes model, a k-means model, a k-nearest neighbors model, a principal component analysis, a Markov model, and any combinations thereof.

3. The computer-implemented method of claim 2, wherein the regression model comprises a multivariate survival model or a multivariate temporal response function model (mTRF model), wherein the mTRF model comprises independent variables corresponding to the multidimensional medical data.

4. The computer-implemented method of claim 2, wherein the generative neural network comprises a conditional generative adversarial network (cGAN), the cGAN comprising one or more conditions corresponding to the multidimensional medical data.

5. The computer-implemented method of claim 1, wherein the baseline multidimensional medical data is collected for the patient within a threshold time period from an index date corresponding to when the patient was diagnosed with HCM.

6. The computer-implemented method of claim 1, wherein the multidimensional medical data comprises at least one of:
medical imaging data;
a cardiac measurement;
clinical data;
electrocardiogram data;
a laboratory test result;
genomic data; or
a functional test result.

7. The computer-implemented method of claim 1, wherein:
the patient has a New York Health Association (NYHA) class assessment of class I; and
the predicted probability comprises a predicted probability of the HCM in the patient progressing to a NYHA class assessment of class II or higher by the threshold date.

8. The computer-implemented method of claim 1, wherein:
the patient comprises a New York Health Association (NYHA) class assessment of early-stage class II; and
the predicted probability comprises a predicted probability of the HCM in the patient progressing to a NYHA class assessment of class III or higher by the threshold date.

9. The computer-implemented method of claim 1, wherein the predicted probability comprises a predicted probability of at least one of:
the HCM in the patient transitioning from non-obstructed HCM to obstructed HCM by the threshold date; or
initiation of an HCM therapy by the threshold date.

10. The computer-implemented method of claim 1, wherein the predicted probability comprises a predicted probability in the patient experiencing a cardiovascular event comprising at least one of:
a cardiovascular-related hospitalization;
a new diagnosis of atrial fibrillation;
an episode of heart failure requiring treatment;
an episode of lethal ventricular arrhythmia leading to sudden cardiac arrest;
an appropriate implantable cardioverter defibrillator shock;
a transient ischemic attack;
a stroke;
death;
an acute myocardial infarction;
a worsening in pVO2; or
a worsening in LVOT gradient.

11. The computer-implemented method of claim 1, wherein the operations further comprise:
receiving follow-up multidimensional medical data for the patient, the follow-up multidimensional medical data collected for the patient during a follow-up visit;
extracting follow-up features from the follow-up multidimensional medical data; and
processing, using one or more trained ML medical prognosis models, the extracted features to predict the probability of experiencing the progression of HCM by the threshold date is further based on processing, using the one or more trained ML medical prognosis models, the extracted follow-up features.

12. The computer-implemented method of claim 1, wherein the operations further comprise:
determining that the predicted probability satisfies a threshold probability value; and
based on determining that the predicted probability satisfies the threshold probability value, selecting the patient for inclusion in a clinical trial.

13. The computer-implemented method of claim 1, wherein the operations further comprise:
determining that the predicted probability satisfies a threshold probability value; and
based on determining that the predicted probability satisfies the threshold probability value, treating the patient with a cardiac myosin inhibitor.

14. A computer-implemented method executed on data processing hardware that causes the data processing hardware to perform operations comprising:
for each particular patient of a plurality of patients diagnosed with hypertrophic cardiomyopathy (HCM) and satisfying an inclusion criterion:
obtaining corresponding baseline training data comprising baseline multidimensional medical data spanning multiple different modalities and collected for the particular patient within a threshold time period from a corresponding index date assigned to the particular patient; and
obtaining corresponding follow-up training data comprising:
follow-up multidimensional medical data spanning multiple different modalities and collected for the particular patient after the corresponding index date; and
a corresponding ground-truth clinical outcome; and
training one or more machine-learning (ML) medical prognosis models on the corresponding baseline training data and the corresponding follow-up training data to teach the one or more ML medical prognosis models to predict the corresponding ground-truth clinical outcomes.

15. The computer-implemented method of claim 14, wherein:
each patient of the plurality of patients comprises a first New York Health Association (NYHA) class assessment at the index date; and
the corresponding ground-truth clinical outcome for a particular patient comprises a progression of the HCM in the patient to a second NYHA class assessment higher than the first NYHA class assessment.

16. The computer-implemented method of claim 14, wherein a corresponding ground-truth clinical outcomes comprises at least one of:
a transition from non-obstructed HCM to obstructed HCM by a threshold date; or
an initiation of an HCM therapy by the threshold date.

17. The computer-implemented method of claim 14, wherein a corresponding ground-truth clinical outcome comprises experiencing a cardiovascular event by a threshold date, the cardiovascular event comprising at least one of:
a cardiovascular hospitalization;
a new diagnosis of atrial fibrillation;
an episode of heart failure requiring treatment;
an episode of lethal ventricular arrhythmia leading to sudden cardiac arrest;
an appropriate implantable cardioverter defibrillator shock;
a transient ischemic attack;
a stroke;
death;
an acute myocardial infarction;
a worsening in pVO2; or
a worsening in LVOT gradient.

18. The computer-implemented method of claim 14, wherein multidimensional medical data spanning multiple different modalities comprises at least one of:
medical imaging data;
a cardiac measurement;
clinical data;
electrocardiograms data;
a laboratory test result;
genomic data; or
a functional test result.

19. The computer-implemented method of claim 14, wherein training the one or more ML medical prognosis models on the corresponding baseline training data and the corresponding follow-up training data obtained for a particular patient comprises, for each respective modality of the multiple different modalities:
extracting, from the corresponding baseline training data, baseline features associated with the respective modality;
extracting, from the follow-up corresponding training data, follow-up features associated with the respective modality; and
training, a respective modality-specific ML medical prognosis model, on the baseline features and the follow-up features associated with the respective modality.

20. The computer-implemented method of claim 14, wherein, for at least one particular patient of the plurality of patients:
obtaining the corresponding baseline training data and the corresponding follow-up training data comprises accessing, via a federated data access technique, a local storage device that stores the corresponding baseline training data and the corresponding follow-up training data for the particular patient, the local storage device controlled by an owner of the corresponding baseline training data and the corresponding follow-up training data; and
training the one or more ML medical prognosis models on the corresponding baseline training data and the corresponding follow-up training data comprises training the one or more prognosis models by processing the corresponding baseline training data and the corresponding follow-up training accessed from the local storage device locally on a respective worker node controlled with the owner of the corresponding baseline training data and the corresponding follow-up training data.
